(19) United States
(12) Patent Application Publication
(10) Pub. No.: US 2023/0078506 A1
(43) Pub. Date: Mar. 16, 2023

(54) AUTOMATIC SYNCHRONIZATION FOR MEDICAL VENTILATION

(71) Applicant: Covidien LP, Mansfield, MA (US)

(72) Inventors: Nancy F. DONG, San Marcos, CA (US); Gabriel SANCHEZ, Valley Center, CA (US); Kun LI, San Diego, CA (US)

(73) Assignee: Covidien LP, Mansfield, MA (US)

(21) Appl. No.: 17/890,957

(22) Filed: Aug. 18, 2022

Related U.S. Application Data
(60) Provisional application No. 63/244,939, filed on Sep. 16, 2021.

(51) Int. Cl. A61M 16/00

(52) U.S. Cl. CPC A61M 16/022; A61M 16/0057; A61M 2016/0018

(57) ABSTRACT

Systems and methods for automatically improving patient-ventilator synchronization, including a method, performed by a ventilator, for automatic synchrony adjustment in medical ventilation. The method may include delivering positive pressure during a first inhalation phase; cycling to a first exhalation phase at an end of the first inhalation phase according to a cycling sensitivity; and at an end of the first exhalation phase, triggering a second inhalation phase. The method may also include during at least one of the first exhalation phase or the second inhalation phase, detecting a cycling-related asynchrony event; in response to the detecting, automatically adjusting the cycling sensitivity without additional user input; delivering positive pressure during the second inhalation phase; and cycling from the second inhalation phase to a second exhalation phase according to the adjusted cycling sensitivity.

### CROSS-REFERENCE TO RELATED APPLICATIONS

This application claims the benefit of U.S. Provisional Application No. 63/244,939 filed Sep. 16, 2021, entitled “Automatic Synchronization for Medical Ventilation,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

In some instances, the breathing gases delivered to the patient may not be synchronous with the patient's own breathing effects. Patient-ventilator asynchrony occurs when the initiation and/or termination of mechanical breath is not in time agreement with the initiation and termination of neural inspiration. Such patient-ventilator asynchrony is a frequent issue in ventilated patients and it is typically uncomfortable for the patient. In addition, patient-ventilator asynchrony may have an impact on patient outcomes. A list of adverse effects associated with poor patient-ventilator interaction includes: a higher or wasted work of breathing; patient discomfort; alveolar overdistention and lung injury; increased need of sedation; prolonged mechanical ventilation; longer hospital stays; and possibly higher mortality.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for automatically improving patient-ventilator synchronization. In an aspect, the technology relates to a method, performed by a ventilator, for automatic synchrony adjustment in medical ventilation. The method includes delivering positive pressure during a first inhalation phase; cycling to a first exhalation phase at an end of the first inhalation phase according to a cycling sensitivity of the ventilator; and at an end of the first exhalation phase, triggering a second inhalation phase. The method further includes during at least one of the first exhalation phase or the second inhalation phase, detecting a cycling-related asynchrony event; in response to the detecting, automatically adjusting the cycling sensitivity without additional user input; delivering positive pressure during the second inhalation phase; and cycling from the second inhalation phase to a second exhalation phase according to the adjusted cycling sensitivity.

In an example, the cycling-related asynchrony event is detected during the first exhalation phase. In another example, the cycling-related asynchrony event is detected during the second inhalation phase. In a further example, the cycling-related asynchrony event is one of a premature-cycling event or a double-triggering event, and adjusting the cycling sensitivity decreases the cycling sensitivity. In yet another example, the cycling-related asynchrony event is a delayed-cycling event, and adjusting the cycling sensitivity increases the cycling sensitivity. In still another example, the cycling-related asynchrony event is detected based on a slope of a net flow crossing zero during the first exhalation phase. In still yet another example, the cycling-related asynchrony event is detected based on at least one of a slope of a net flow exceeding a slope threshold during the first exhalation phase, or a second slope of a net flow exceeding a slope threshold during the first exhalation phase. In another example, the method further includes receiving a user input to activate an automated synchronization mode.

In another aspect, the technology relates to a ventilator for providing automatic synchrony adjustment in medical ventilation. The ventilator includes a pressure generating system; a processor; and memory storing instructions that, when executed by the processor, cause the ventilator to perform a set of operations. The operations include delivering positive pressure during a first inhalation phase; cycling to a first exhalation phase at an end of the first inhalation phase according to a cycling sensitivity of the ventilator; at an end of the first exhalation phase, triggering a second inhalation phase; during at least one of the first exhalation phase or the second inhalation phase, detecting a cycling-related asynchrony event; in response to the detecting, during the first exhalation phase, automatically adjusting the cycling sensitivity without additional user input; delivering positive pressure during the second inhalation phase; and cycling from the second inhalation phase to a second exhalation phase according to the adjusted cycling sensitivity.

In an example, the cycling-related asynchrony event is one of a premature-cycling event or a double-triggering event, and adjusting the cycling sensitivity decreases the cycling sensitivity. In another example, the cycling-related asynchrony event is a delayed-cycling event, and adjusting the cycling sensitivity increases the cycling sensitivity. In still another example, the cycling-related asynchrony event is detected based on a slope of a net flow crossing zero during the first exhalation phase. In yet another example, the cycling-related asynchrony event is detected based on a second slope of a net flow exceeding a slope threshold during the first exhalation phase.

In another aspect, the technology relates to a method, performed by a ventilator, for automatic synchrony adjustment in medical ventilation. The method includes triggering a first inhalation phase according to a triggering sensitivity of the ventilator; delivering positive pressure during a first inhalation phase; cycling to a first exhalation phase at an end of the first inhalation phase; during the first exhalation phase, detecting a first triggering-related asynchrony event; in response to detecting the first triggering-related asynchrony event, during the first exhalation phase automatically adjusting the triggering sensitivity without additional user input; at an end of the first exhalation phase, triggering a second inhalation phase according to the adjusted triggering sensitivity; and delivering positive pressure during the second inhalation phase.

In an example, the first triggering-related asynchrony event is a missed-triggering event, and adjusting the triggering sensitivity increases the triggering sensitivity. In another example, the first triggering-related asynchrony event is an auto-triggering event, and adjusting the triggering sensitivity decreases the triggering sensitivity. In yet another example, the first triggering-related asynchrony event is detected based on a local minimum and a local maximum of a slope of net flow signal. In still another example, the first triggering-related asynchrony event is detected based on a local minimum and a local maximum of a slope of a derivative of an intrapleural pressure signal. In yet another example, adjusting the triggering sensitivity adjusts the triggering sensitivity by a first amount, and the method further includes: cycling from the second inhalation phase to a second exhalation phase; during the second exhalation phase, detecting a second triggering-related asynchrony event; and based on detecting the second triggering-related asynchrony event, re-adjusting the adjusted triggering sensitivity by a second amount that is different than the first amount. In still yet another example, the first triggering-related asynchrony event is an auto-triggering event, the second triggering-related asynchrony event is a missed-triggering event; and the first amount is less than the second amount.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example of a medical ventilator connected to a human patient.

FIG. 2 depicts an example method for ventilator auto-synchronization.

FIG. 3 depicts example plots for an example of an ineffective effort (i.e., missed triggering (MT)) asynchrony event.

FIGS. 4A-4B depict example plots for an example of an auto-triggering asynchrony event.

FIGS. 5A-5B depict example plots for an example of a double-trigger asynchrony event.

FIGS. 6A-6B depict example plots for an example of a premature cycling asynchrony event.

FIGS. 7A-7B depict example plots for an example of a delayed cycling asynchrony event.

FIG. 8 depicts an example method for automatic synchrony adjustment in medical ventilation.

FIG. 9 depicts another example method for automatic synchrony adjustment in medical ventilation.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

Medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

As discussed briefly above, patient-ventilator asynchrony is a frequent problem that results in patient discomfort and may have negative impacts on patient outcomes. The various types of major patient-ventilator asynchrony include (1) ineffective effort (i.e., missed triggering (MT); (2) auto triggering (AT); (3) double triggering (DT); (4) premature cycling (PC); and (5) delayed cycling (DC).

Each of the patient asynchrony types results from the ventilator triggering and/or cycling at an improper time that is out of synchronization with the patient's own breathing efforts. Some current triggering and cycling methods include a flow method, a pressure method, and an IE Sync method or signal distortion method. All these triggering and cycling methods include triggering and cycling sensitivity settings that at least partially control when triggering and cycling occurs. In some scenarios, when the patient is first connected to the ventilator, a clinician uses default triggering and cycling settings then fine-tunes them based on the patient's initial conditions and other ventilator settings. However, during ventilation, patient-ventilator asynchrony can result due to variability of patient conditions and breathing patterns with the fixed triggering/cycling sensitivity settings.

The present technology provides for, among other things, improvements to patient synchronization during ventilation by automatically adjusting ventilator triggering sensitivity and/or cycling sensitivity based on the detected types of patient-ventilator asynchronies. This automated synchronization (“AutoSync”) technology includes asynchrony detection algorithms to reliably and accurately detect the patient-ventilator asynchrony and classify the type of asynchrony. The asynchrony detection algorithms may be continuously performed during ventilation, and the triggering and/or cycling criteria may be automatically adjusted to maintain ventilator synchrony in substantially real time. For instance, changes to sensitivity values may be achieved on a breath-to-breath basis. As a result, the present technology for improved ventilator synchronization without the need for additional hardware or accessories and further reduces the need for user settings or interactions with the ventilator.

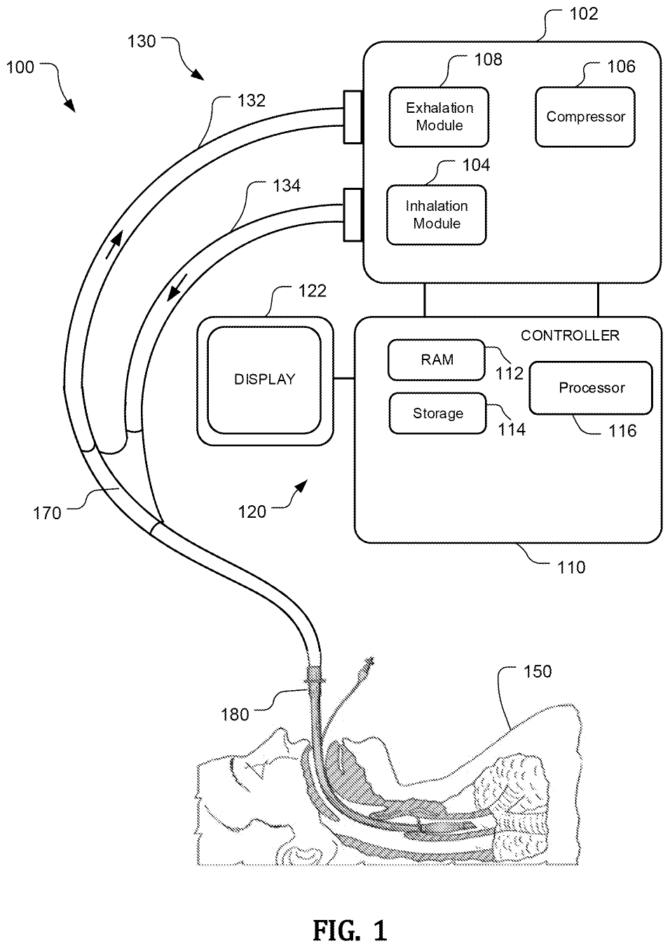

FIG. 1 is a diagram illustrating an example of a medical ventilator 100 connected to a human patient 150. The ventilator 100 may provide positive pressure ventilation to the patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. For instance, the memory 112 may store instructions that, when executed by the processor 116, cause the ventilator 100 to perform operations such as the operations described herein. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

As each patient may require a different ventilation strategy, modern ventilators can be customized for the particular needs of an individual patient. For example, several different ventilator modes, breath types, and/or settings have been created to provide clinically appropriate ventilation for patients in various different scenarios, such as mandatory ventilation modes and assist control ventilation modes. Assist control modes (also referred to herein as “spontaneous modes”) allow a spontaneously breathing patient to trigger inspiration during ventilation. In a spontaneous or assisted mode of ventilation, the ventilator begins (triggers) inspiration upon the detection of patient demand or patient effort to inhale. The ventilator ends inspiration and begins expiration (cycles to expiration) when a threshold is met or when a patient demand or effort for exhalation is detected.

In some triggering modes, a patient's inspiratory trigger is detected based on the magnitude of deviations (deviations generated by a patient's inspiratory effort) of a measured parameter from a determined baseline. For example, in flow triggering, the patient's inspiration effort is detected when the measured patient exhalation flow value drops below a flow threshold or baseline (i.e. the base flow) by a set amount (based on the triggering sensitivity). In pressure triggering, the patient's inspiration effort is detected when the measured expiratory pressure value drops below a pressure baseline (for example, the set PEEP value) by a set amount (based on triggering sensitivity). Another parameter that can be used for triggering is a derived signal, such as an estimate of the intrapleural pressure of the patient and/or the derivative of the estimate of the patient's intrapleural pressure. The term “intrapleural pressure,” as used herein, refers generally to the pressure exerted by the patient's diaphragm on the cavity in the thorax that contains the lungs, or the pleural cavity. The derivative of the intrapleural pressure value will be referred to herein as a “Psync” value that has units of pressure per time. An example of triggering and cycling based on the Psync value is provided in U.S. patent application Ser. No. 16/411,916 (“the '916 Application”), titled “Systems and Methods for Respiratory Effort Detection Utilizing Signal Distortion” and filed on May 14, 2019, which is incorporated herein by reference in its entirety. That triggering mode discussed in the '916 Application is referred to as the “signal distortion” triggering mode. As discussed in the '916 Application, the signal distortion triggering mode may operate on the Psync signal or other signals, such as flow or pressure.

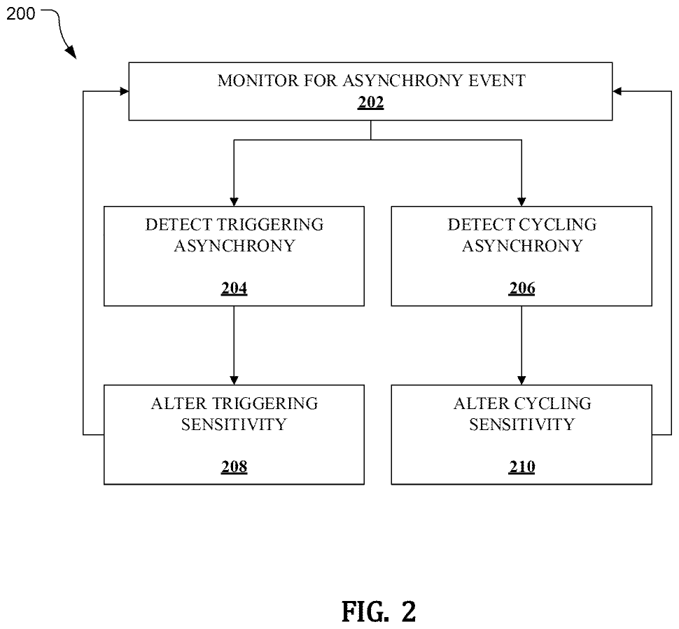

FIG. 2 depicts an example method 200 for ventilator auto-synchronization. At operation 202, the ventilator monitors for an asynchrony event. Monitoring for an asynchrony event may include continuously, or substantially continuously, performing asynchrony detection algorithms discussed herein (e.g., continuously evaluating the criteria discussed herein). Such performance or execution of the algorithms may include analyzing various waveforms of ventilation characteristics and other values calculated or derived therefrom.

Two types of asynchrony events may be detected when monitoring for an asynchrony event. The first type of asynchrony event is a triggering-related asynchrony that occurs when the ventilator triggers an inspiratory phase either too early or too late. The inspiratory phase may also be referred to as an inhalation phase. The second type of asynchrony event is a cycling-related asynchrony event that occurs when the ventilator cycles from an inspiratory phase to an expiratory phase either too early or too late. The expiratory phase may also be referred to as an exhalation phase.

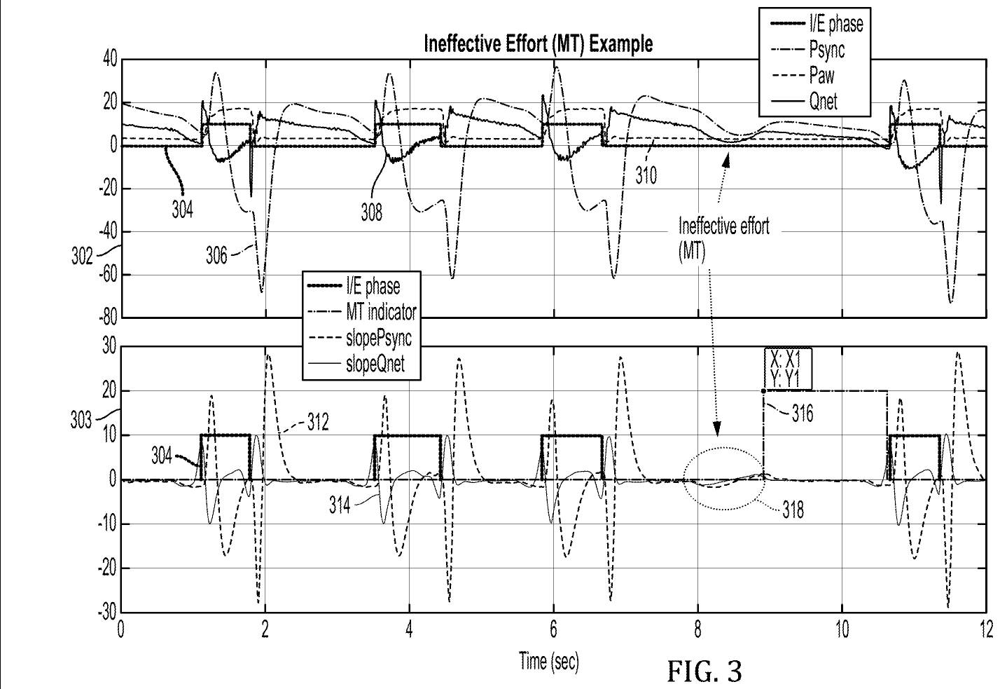

Example triggering-related asynchrony events include an ineffective effort (i.e., missed triggering (MT)) event and an auto-triggering (AT) event. Example cycling-related asynchrony events include a premature cycling event, a delayed cycling event, and a double triggering event. Additional details regarding these different types of asynchrony events and examples of how to detect such events is described further below with reference to FIGS. 3-10.

While monitoring for asynchrony events at operation 202, a triggering-asynchrony may be detected at operation 204. In response to detecting a triggering-related asynchrony event, the ventilator may automatically alter a triggering sensitivity at operation 208. Altering the ventilator's triggering sensitivity may be accomplished by adjusting a value associated with the triggering sensitivity. The magnitude and the direction of the change of the triggering sensitivity value may be based on the type of asynchrony event that is detected. For example, where a missed triggering (MT) event is detected and where the triggering sensitivity value is a triggering threshold, the triggering threshold or sensitivity value is decreased by a first interval amount. In some examples, decreasing the triggering threshold or sensitivity value results in the ventilator being more sensitive for triggering purposes. In other words, a smaller effort from the patient will cross the threshold and trigger an inspiratory phase. Such a change that allows a smaller or weaker patient effort to trigger a breath may be referred to as raising the triggering sensitivity or making the ventilator more sensitive or responsive to triggering. Thus, because a missed trigger event was detected, the ventilator is made to be more sensitive to trigger events and responds sooner to detect a trigger (e.g., by decreasing the triggering sensitivity value such as the triggering threshold). As another example, where an auto-triggering (AT) event is detected and where the triggering sensitivity value is a triggering threshold, the triggering threshold or sensitivity value is increased by a second interval amount (e.g., the ventilator is made less sensitive and a larger breathing effort from the patient is required to cross the threshold and trigger an inspiratory phase). Increasing the triggering threshold may cause the ventilator to be less sensitive or responsive for triggering purposes. In other words, a greater patient effort is needed to trigger a breath. Such a change that that requires a greater patient effort to trigger a breath may be referred to as lowering the ventilator's triggering sensitivity.

As an illustrative example, the triggering sensitivity value may be a flow sensitivity threshold value or setting. The flow sensitivity threshold defines the rate of flow inspired by a patient that triggers the ventilator to deliver a mandatory or spontaneous breath. When the patient inhales and their inspiratory flow exceeds the flow sensitivity threshold, a trigger occurs and the ventilator delivers a breath. Thus, a flow sensitivity threshold that is set too low may result in auto-triggering asynchronies, whereas a flow sensitivity threshold that is set too high may result in missed triggering asynchrony events. In some implementations, the flow sensitivity threshold setting may range between 0.1 liters per minute (lpm) to 20 lpm for an adult or pediatric circuit.

As another example, the triggering sensitivity value may be a pressure sensitivity threshold or setting. The pressure sensitivity threshold or setting sets the pressure drop below baseline (PEEP) required to begin a patient-initiated breath (either mandatory or spontaneous). Lower pressure sensitivity thresholds or settings require less patient effort to initiate a breath. However, fluctuations in system pressure can cause auto-triggering at very low thresholds or settings. In contrast, a high pressure sensitivity setting or threshold may result in missed-triggering asynchrony events. In some implementations, the pressure sensitivity setting may range from 0.1 cmH₂O to 20.0 cmH₂O.

As yet another example, the triggering sensitivity value may be for an I/E Sync or signal distortion triggering mode, where the triggering sensitivity values have a range of −2 to +2. Similar to other types of triggering sensitivity values, an increase of the triggering sensitivity value results in the patient having to provide a stronger effort to trigger the delivery of a breath (e.g., trigger an inspiratory phase). While different types of triggering sensitivity values or settings have been discussed above, it should be appreciated that in different examples and configurations, different types of values or changes may be used to cause the desired adjustment to the ventilator triggering sensitivity.

The first interval amount and the second interval amount by which the triggering sensitivity values is changed may be the same or different values. For instance, a missed triggering event may be a more severe asynchrony event than an auto-triggering event. As such, the first interval amount associated with the missed triggering event may have a greater magnitude than the second interval amount associated with the auto-triggering event. As an example, where a flow sensitivity value is used, the first interval amount associated with the missed triggering event may be 0.2 lpm or 0.5 lpm, whereas the second interval amount associated with the auto-triggering event may be 0.1 lpm or 0.3 lpm.

After the triggering sensitivity is altered or adjusted in operation 208, the method 200 flows back to operation 202 where monitoring for additional asynchrony events continues. While monitoring for an asynchrony event in operation 202, a cycling-related asynchrony event may be detected at operation 206. In response to detecting a cycling-related asynchrony event, a cycling sensitivity of the ventilator may be altered or adjusted in operation 210. Altering the ventilator's cycling sensitivity may be accomplished by adjusting a value associated with the cycling sensitivity. The magnitude and the direction of the change of the cycling sensitivity value may be based on the type of asynchrony event that is detected. For example, for a premature cycling event or double triggering event, the cycling sensitivity threshold value is decreased to make the ventilator less sensitive to cycling (e.g., cycling occurs later and/or the inspiratory phase duration is longer). Such a change that causes cycling to occur later may be referred to as lowering the cycling sensitivity of the ventilator to make the ventilator respond less quickly to detect a cycling event. As another example, for a delayed cycling event, the cycling sensitivity threshold value is increased to make the ventilator more sensitive to cycling (e.g., cycling occurs earlier or the inspiratory phase duration is shorter). Such a change that causes cycling to occur earlier may be referred to as raising the cycling sensitivity of the ventilator to make the ventilator respond more quickly to detect a cycling event.

In some examples, the cycling sensitivity threshold or value may be represented as a percentage of peak flow. For instance, the cycling sensitivity value may define the percentage of the measured peak inspiratory flow at which the ventilator cycles from inspiration to exhalation in spontaneous breath types. In some examples, the range for such percentages may be between 1% and 80%. When inspiratory flow falls to the level defined by the cycling sensitivity value, cycling occurs and exhalation begins. In such an example, a higher cycling sensitivity value results in a shorter the inspiratory time (e.g., a higher ventilator cycling sensitivity). In some modes, the cycling sensitivity value may also be presented as a flow value of liters per minute or as a unitless range of −2 to +2 (or some other similar range). Depending on the type of cycling sensitivity value, an increase and/or decrease to the value may cause an increase and/or decrease to the ventilator cycling sensitivity. The amount that the cycling sensitivity changes may also be dependent on the type of asynchrony event detected. For example, for a delayed-cycling event, the cycling sensitivity may be increased by 5%, whereas for a premature cycling or double-triggering event, the cycling sensitivity may be decreased by 10%.

After the cycling sensitivity value is altered or adjusted in operation 210, the method 200 flows back to operation 202 where monitoring for additional asynchrony events continues. Method 200 may continue to loop for as long as the patient is being mechanically ventilated.

FIG. 3 depicts example plots for an example of an ineffective effort (i.e., missed triggering (MT)) asynchrony event. More specifically, FIG. 3 includes an upper plot 302 and a lower plot 303. The upper plot 302 includes an I/E phase signal 304, a Psync signal 306, a net flow (Qnet) signal 308, and an airway pressure signal (Paw) 310. The lower plot 303 includes the I/E phase signal 304, a slope of the Psync signal (slopePsync) 312, and a slope of the net flow signal (slopeQnet) 314. The lower plot 303 also includes a missed trigger (MT) signal 316.

The TIE phase signal 304 is a binary signal that is either high or low. When the I/E phase signal 304 is high (e.g., non-zero in the example depicted), the ventilator is in an inspiratory phase and is delivering a breath to the patient. When the I/E phase signal 304 is low, the ventilator is an expiratory phase and allowing the patient to exhale. The Psync signal represents the derivative of a calculated intrapleural pressure value and has units of pressure per time, as discussed above and in the '916 Application. The airway pressure signal 310 represents the pressure of the airway and has units of pressure. The airway pressure signal 310 may be the mean airway pressure and may be measured at the wye or at other locations within the breathing circuit and/or ventilator. The net flow (Qnet) signal 308 represents the net flow, which may be determined based on an inspiratory flow (Qinsp) measurement and an exhalation flow (Qexh) measurement. The Qnet signal 308 in FIG. is calculated as follows: Qnet=Qexh−Qinsp). The missed trigger (MT) signal 316 is a binary signal that goes high/true when a missed trigger (MT) asynchrony event has been detected.

The slope of the Psync signal (slopePsync) 312 represents a change in the Psync signal over time, and the slopePsync signal 312 may have units of pressure per time squared (e.g., cmH₂O/s²). The slopePsync signal may be determined or calculated by subtracting a current Psync value from a prior Psync value. For example, the slopePsync signal 312 may be calculated using the following Equation 1:

slopePsync=Psync(k)−Psync(k−α) (Eqn. 1)

In Equation 1, k is a discrete time index and may represent a current measurement cycle or control cycle. The constant α represents a number of prior measurement cycles (or other time index as appropriate). As an example, measurements may be made or calculated by the ventilator, at discrete measurement cycles or control cycles, such as 2 milliseconds (ms), 5 ms, 8 ms, 10 ms, 20 ms, etc. The constant α is an integer and thus represents a number of control cycles and is effectively equal to a time (e.g., a multiplied by the control cycle duration equals a time duration). The constant α may be between 2-20, depending on the length of the control cycle. The ultimate duration prior to current control cycle may be between 20 ms and 100 ms.

The slope of slope of the net flow signal (slopeQnet) 314 represents a change in the net flow (Qnet) signal over time, and the slopeQnet signal 314 may have units of flow per time (e.g., Liter/min²). The slopeQnet signal may be determined or calculated by subtracting a current Qnet value from a prior Qnet value. For example, the slopeQnet signal 314 may be calculated using the following Equation 2:

slopeQnet=Qnet(k)−Qnet(k−α) (Eqn. 2)

Similar to Equation 1, in Equation 2, k represents a current measurement cycle or control cycle and α is a constant that represents a number of prior measurement cycles.

In the plots of FIG. 3, an occurrence of a missed trigger asynchrony event is highlighted by the circle 318. Ineffective efforts that cause missed triggers include a patient's efforts that fail to trigger the ventilator, which are the most common form of patient ventilator asynchrony. Events of ineffective efforts are identified in 38% of patients with prolonged mechanical ventilation and were associated with increased mortality. During mechanical exhalation (e.g., an expiratory phase), if a patient's respiratory effort occurs, the effort pulls both Qnet and Psync down (e.g., lower or more negative) until the mechanical inspiration is triggered. But if the ventilator misses the effort, the Qnet and Psync is released (e.g., moves upward) with the release of patient's inspiratory effort. This phenomenon can be seen in the highlighted circle 318. As such, detection of such a dip and rise in Qnet and Psync during an exhalation phase may indicate a missed trigger asynchrony event has occurred. For instance, for a missed trigger to be detected, Qnet and Psync must both be negative and positive at some point during the exhalation phase of a breath. Other cardiogenic events may also create similar phenomena, so additional criteria may also be evaluated to distinguish ineffective efforts from those other cardiogenic events.

In some examples, the following criteria may be used to determine whether a missed-trigger asynchrony event has occurred. Specifically, if the following four criteria are met, a missed-trigger asynchrony event is declared and the missed trigger (MT) signal 316 goes to high/true:

Example Ineffective Effort (Missed-Triggering (MT)) Detection Criteria:
(1) breath phase is exhalation phase; and
(2) exhalation time has passed a set duration; and
(3) a local minimum of slopePsync below zero is detected, a local minimum of slopeQnet below zero is detected, a local maximum of slopePsync above zero is detected after the local minimum of slopePsync, and a local maximum of slopeQnet above zero is detected after the local minimum of slopePsync; and
(4) the value of local minimums of slopePsync and slopeQnet are both smaller (more negative) than −β1 and the local maximums of slopePsync and slopeQnet are both greater (more positive) than β2.

In some examples, the set duration in Criterion 2 may be between 0-500 ms, such as 50 ms, 100 ms, 200 ms, 250 ms, etc. The constants β1 and β2 in Criterion 4 are threshold may be used to distinguish the missed trigger event due to an ineffective effort from other types of cardiogenic events. The constants β1 and β2 may within the range of 0.2-1.0, such as 0.2, 0.4, 0.5, 0.6, 0.8, etc. where slopeQnet may have units of Liter/min² and slopePsync may have units of cmH₂O/s².

The above criteria may be evaluated during the same exhalation phase for which the asynchrony event occurs. In such examples, upon the missed trigger asynchrony event being detected, the triggering sensitivity may be altered prior to the next triggering event (e.g., prior to the next breath). In other examples, the triggering sensitivity is altered prior to a second inspiratory phase after the occurrence of the event being detected. The detection and adjustment of the triggering sensitivity may all be performed without additional input or interaction from the clinician or other user.

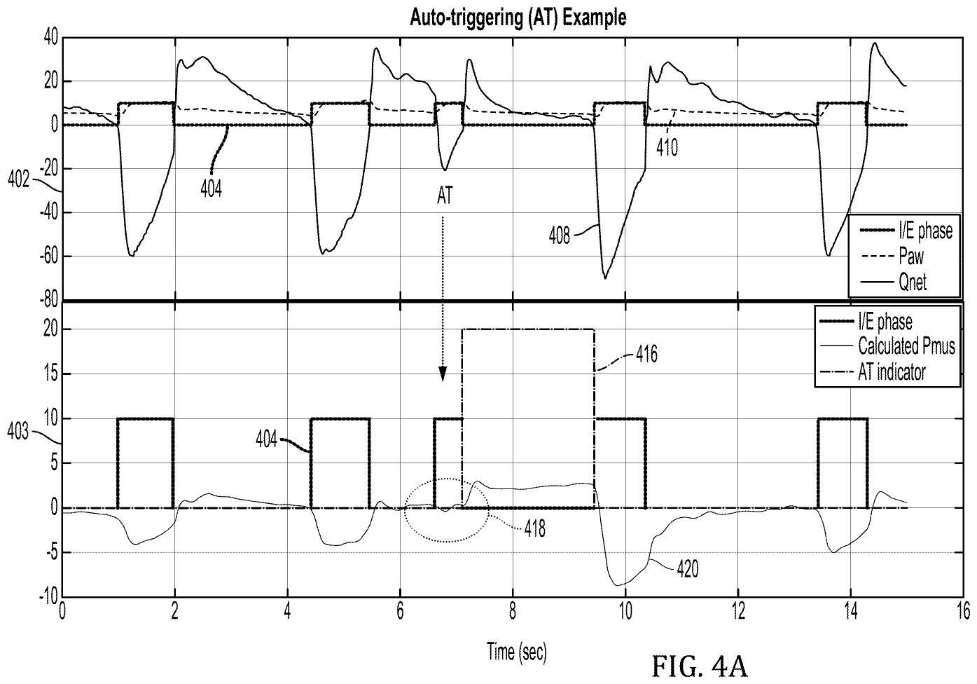

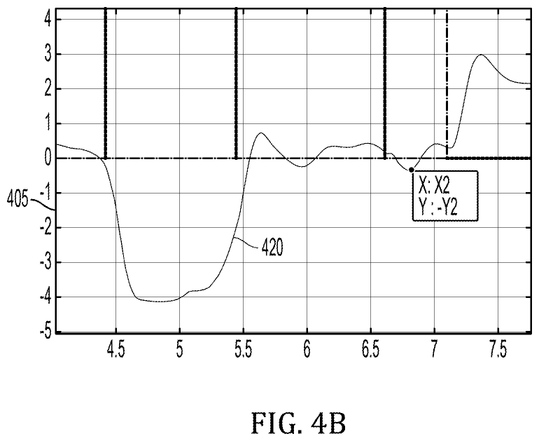

FIGS. 4A-4B depict example plots for an example of an auto-triggering asynchrony event. More specifically, FIGS. 4A-4B include an upper plot 402, a lower plot 403, and an enlarged portion 405 of the lower plot 403. The upper plot 402 includes the I/E phase signal 404, the net flow (Qnet) signal 408, and the airway pressure signal (Paw) 410. The lower plot 403 (and the enlarged portion 405) include the I/E phase signal 404, a calculated Pmus signal 420, and an auto-triggering (AT) signal 416. The auto-triggering (AT) signal 416 is a binary signal that goes high/true when an auto-triggering (AT) asynchrony event has been detected.

The calculated Pmus signal represents the pressure generating capability of the inspiratory muscles over time. Rather than a directly measured Pmus value, the Pmus may be calculated using the equation of motion. For example, the calculated Pmus value may be calculated according to the following Equation 3:

Pₘᵤₛ=P_{αw}−R_{αw}*Q_{lung}−(1/C_{lung})*V_{lung}−Iᵣₛ*\dot{Q}_{lung} (Eqn. 3)

In Equation 3, where Q_{lung}=lung flow, which can be calculated by utilizing the ventilator internal flow sensor measurements; R_{αw}=airway resistance; V_{lung}=lung volume, which is integration of Q_{lung}; C_{lung}=lung compliance; Iᵣₛ=respiratory system inertance, which represents the inertia of the flow; and \dot{Q}_{lung}=acceleration of the flow throughout patient's airway.

In the plots of FIGS. 4A-4B, an occurrence of an auto-triggering asynchrony event is highlighted by the circle 418. Auto-triggering occurs when the ventilator is triggered and delivers a new breath in the absence of patient's inspiratory effort, which is a common phenomenon during assisted ventilation. These additional breaths may lead to hyperinflation, respiratory alkalosis, hyperventilation, and diaphragmatic dysfunction. To detect an auto-triggering event, the Pmus signal may be analyzed. In a properly triggered breath, the Pmus signal drops substantially below zero, indicating a large patient effect. In an auto-triggered breath, the Pmus value may be less negative (e.g., more shallow) as compared to the Pmus of prior breaths.

In some examples, the following criteria may be used to determine whether an auto-triggering asynchrony event has occurred. Specifically, if the following three criteria are met, an auto-triggering asynchrony event is declared and the auto-triggering asynchrony signal 416 goes to high/true:

Example Auto-Triggering Asynchrony Detection Criteria:
(1) at the start of exhalation phase;
(2) a local minimum of calculated Pmus in the prior inspiratory phase is greater than a threshold (γ) (e.g., is not more negative than the threshold (γ)); and
(3) local minimum of calculated Pmus in the prior inspiratory phase is greater than δ*(Pmusₘₑₐₙ₋ₘᵢₙ).

The threshold (γ) is in Criterion 2 a constant that may be within a range of values. For example, the threshold (γ) may be between −0.25 cmH₂O and −5 cmH₂O, such as −1.0 cmH₂O, −2.0 cmH₂O, −3.0 cmH₂O, etc. The threshold (γ) is used to distinguish patient efforts that are indicative of a breath (e.g., large efforts represented by negative Pmus values with high magnitudes) from those effects that are not indicative of a breath (e.g., small efforts represented by negative Pmus values with small magnitudes).

Criterion 3 is utilized to confirm that the auto-triggering event occurred based on the patient's own breathing pattern for a number of prior breaths. In Criterion 3, δ is a constant between 0.2 and 0.8, such as 0.3, 0.4, 0.5, 0.6, 0.7, etc. The variable Pmusₘₑₐₙ₋ₘᵢₙ represents an average local minimum of Pmus over a number of prior breaths during the inspiratory phase. For example, number of prior breaths may be between 2-10 breaths among other possible numbers of prior breaths.

The above criteria may be evaluated during the exhalation phase following the asynchrony event. In such examples, upon the auto-triggering asynchrony event being detected, the triggering sensitivity may be altered prior to the next triggering event (e.g., prior to the next breath). In other examples, the triggering sensitivity is altered prior to a second inspiratory phase after the occurrence of the event being detected. The detection and adjustment of the triggering sensitivity may all be performed without additional input or interaction from the clinician or other user.

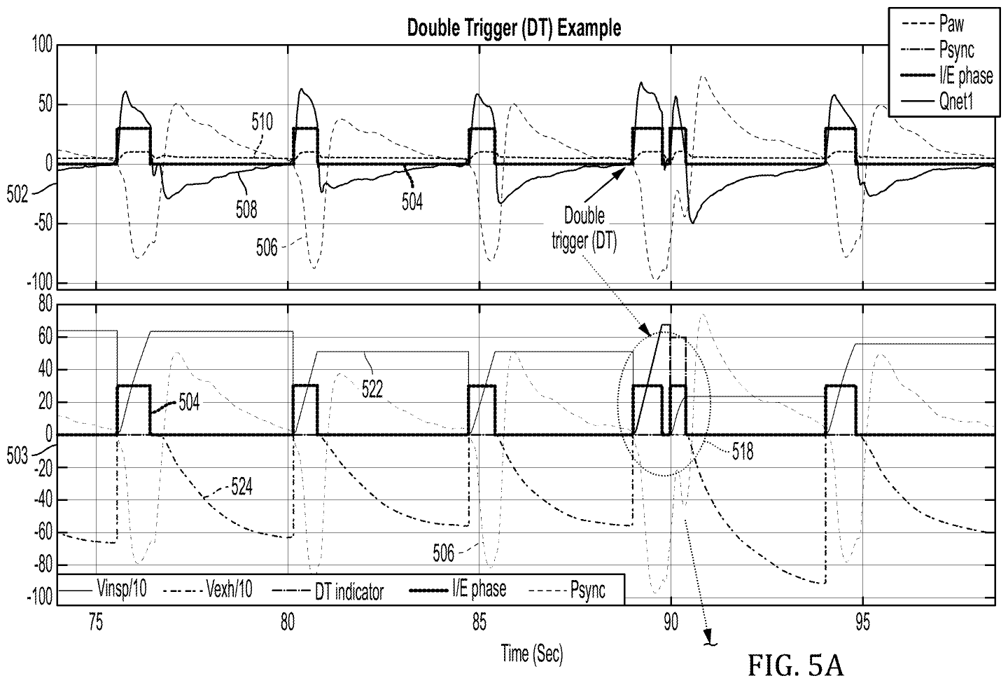

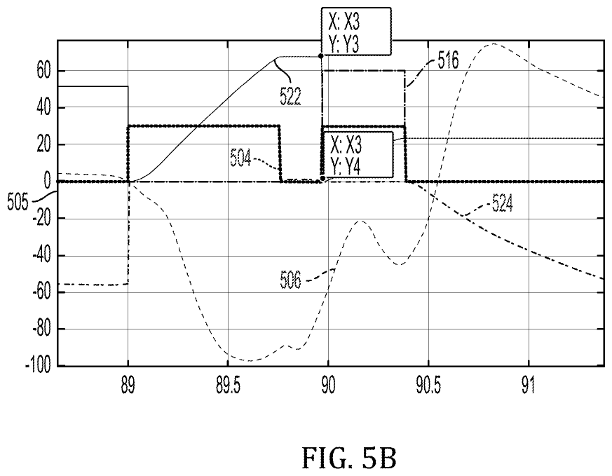

FIGS. 5A-5B depict example plots for an example of a double-trigger asynchrony event. More specifically, FIGS. 5A-5B include an upper plot 502, a lower plot 503, and an enlarged portion 505 of the lower plot 503. The upper plot 502 includes the I/E phase signal 504, a Psync signal 506, a net flow signal (Qnet1) 508, and an airway pressure signal (Paw) 510. The lower plot 503 and the enlarged portion 505 include a the I/E phase signal 504, the Psync signal 506, a 10% inspired volume signal 522, a 10% exhaled volume signal 524, and a double-triggering (DT) signal 516. The double-triggering (DT) signal is a binary signal that goes high/true when an auto-triggering asynchrony event has been detected. Of note, the Qnet1 flow signal 508 is the additive inverse of the Qnet signal described above. For instance, Qnet1=Qinsp−Qexh.

In the plots of FIGS. 5A-5B, an occurrence of a double-triggering asynchrony event is highlighted by the circle 518. Double triggering refers to the occurrence of two consecutive inspirations with one inspiratory effort. Double-triggering generally occurs when respiratory drive is high, ventilator support is insufficient, or neural inspiratory time is longer than ventilator delivered inspiratory time. Double-triggering may provoke high tidal volume (up to twice of the set value) putting the patient at risk of ventilator-induced lung injury and ventilator-induced diaphragmatic dysfunction. During a double-triggered event, the patient's respiratory effort continues even during the exhalation phase and the flow is pulled to the lungs, which is reflected in a negative value of Psync. In addition, during a double-triggering event, the exhalation phase is generally short and the expired volume is much smaller compared to the inspired volume since the patient's respiratory effort continues.

In some examples, the following criteria may be used to determine whether a double-triggering asynchrony event has occurred. Specifically, if the following four criteria are met, an auto-triggering asynchrony event is declared and the double-triggering signal 516 goes to high/true:

Example Double-Triggering Asynchrony Detection Criteria:
(1) at the start of inspiratory phase;
(2) the Psync value is always negative during the past exhalation phase;
(3) in the past breath, the expired volume is less than ε*inspired volume (e.g., −Vexh<ε*Vinsp); and
(4) the duration of the past exhalation phase is shorter than a duration threshold (ζ).

The constant ε in Criterion 3 is a fraction less than 1. Thus, Criterion 3 ensures that the expired volume is substantially less than the inspired volume, which is indicative of a double-trigger event. In some examples, the constant ε is between 0.05-0.5, such as 0.1, 0.2, 0.3, 0.4, etc. Criterion 4 confirms that the prior exhalation phase was short, e.g., less than the duration threshold (ζ). In some examples, the duration threshold (ζ) may be between 200-800 ms, such as 300 ms, 400 ms, 500 ms, etc.

The above criteria may be evaluated during the second inspiratory phase of the double triggering asynchrony event. In such examples, upon the double-triggering asynchrony event being detected, the cycling sensitivity may be altered prior to the next cycling event (e.g., prior to the next breath). In other examples, the cycling sensitivity is altered prior to a second exhalation phase after the occurrence of the asynchrony event being detected. The detection and adjustment of the cycling sensitivity may all be performed without additional input or interaction from the clinician or other user.

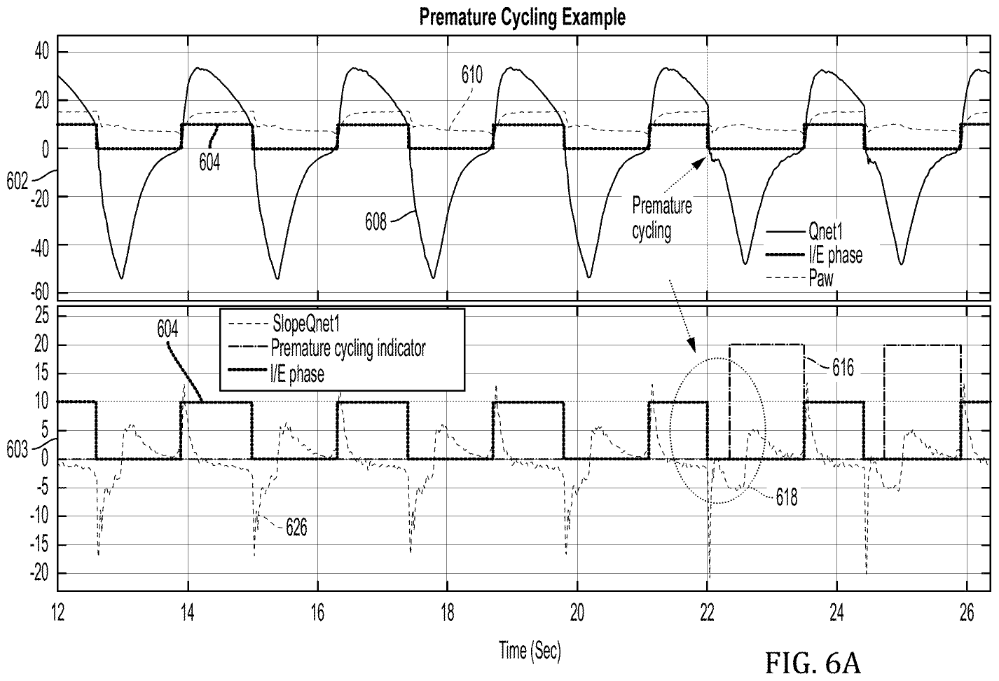

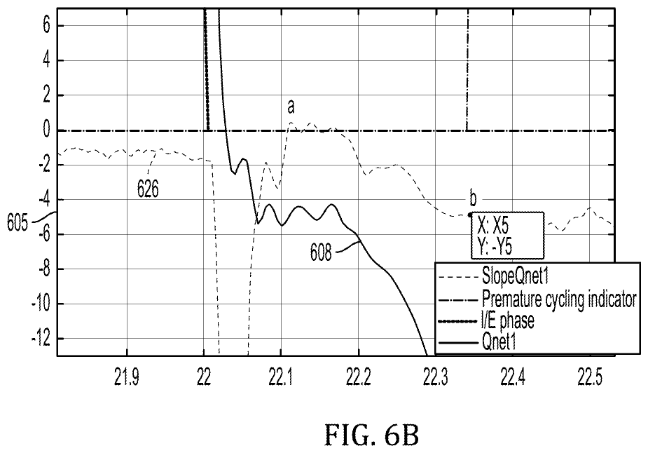

FIGS. 6A-6B depict example plots for an example of a premature cycling asynchrony event. More specifically, FIGS. 6A-6B include an upper plot 602, a lower plot 603, and an enlarged portion 605 of the lower plot 603. The upper plot 602 includes in the I/E phase signal 604, the net flow signal (Qnet1) 608, and the airway pressure signal (Paw) 610. The lower plot 603 includes the I/E phase signal 604, a slope of the net flow signal (slopeQnet1) 626, and a premature-cycling signal 616. The premature-cycling signal is a binary signal that goes high when a premature-cycling asynchrony event has been detected.

The slope of the net flow signal (slopeQnet1) 626 represents a change in the net flow signal (Qnet1) over time. The slopeQnet1 signal may be determined or calculated by subtracting a current Qnet1 value from a prior Qnet1 value. For example, the slopeQnet1 signal 626 may be calculated using the following Equation 4:

slopeQnet1=Qnet1(k)−Qnet1(k−α) (Eqn. 4)

In the plots of FIGS. 6A-6B, an occurrence of a premature-cycling asynchrony event is highlighted by the circle 618. Premature cycling occurs when the exhalation valve opens too early causing mechanical inspiration to last shorter than the neural inspiration. This form of asynchrony is often associated with insufficient ventilation support and also puts the patient at risk for double-triggering. If a breath is prematurely cycled off, the patient's inspiratory effort continues until the neural inhalation ends. Therefore, at the beginning of exhalation (right after premature cycling), the flow is still pulled into the patient's lungs, which results in non-descending flow (Point “a” in the enlarged portion 605). In addition, when the patient's inspiratory effort ends and starts to release, the flow is exhaled out of the lungs. The exhaled flow causes a drop in slopeQnet1, which can be seen in Point “b” in the enlarged portion 605.

In some examples, the following criteria may be used to determine whether a premature-cycling asynchrony event has occurred. Specifically, if the following three criteria are met, premature-cycling asynchrony event is declared and the premature-cycling signal 616 goes to high/true:

Example Premature-Cycling Asynchrony Detection Criteria:
(1) within the first duration threshold (η) of expiration phase;
(2) a positive trending (e.g., negative to positive) zero crossing of the slopeQnet1 signal 626 is detected; and
(3) the slopeQnet1 is less than (e.g., more negative) than a slope threshold (θ).

The constant η in Criterion 1 is a time duration that indicates a short term at the beginning of the expiration phase. In some examples, the constant η may be between 200 ms and 800 ms, such as 300 ms, 400 ms, 500 ms, 600 ms, 700 ms, etc. The slope threshold (θ) is a value to help confirm that exhalation has occurred. In some examples, the slope threshold may be between −2 liter/min² and −10 liter/min², such as −4, −5, −6, −7 liter/min² etc.

The above criteria may be evaluated during the expiration phase following the premature cycling event. In such examples, upon the premature-cycling asynchrony event being detected, the cycling sensitivity may be altered prior to the next cycling event (e.g., prior to the next breath). In other examples, the cycling sensitivity is altered prior to a second exhalation phase after the occurrence of the asynchrony event being detected. The detection and adjustment of the cycling sensitivity may all be performed without additional input or interaction from the clinician or other user.

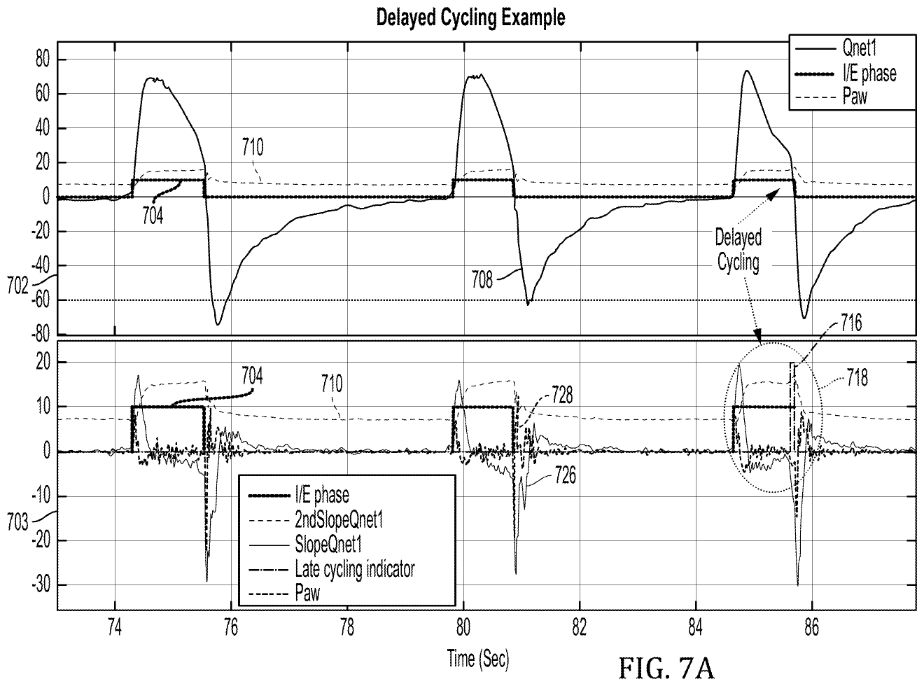

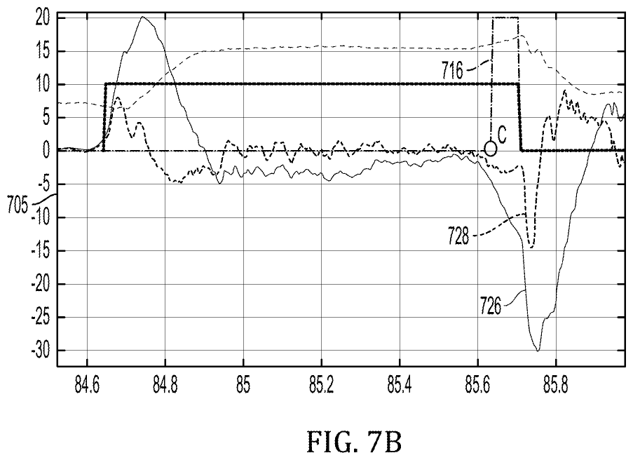

FIGS. 7A-7B depict example plots for an example of a delayed cycling asynchrony event. More specifically, FIGS. 7A-7B include an upper plot 702, a lower plot 703, and an enlarged portion 705 of the lower plot 703. The upper plot 702 includes the I/E phase signal 704, the net flow signal (Qnet1) 708, and the airway pressure signal (Paw) 710. The lower plot 703 and the enlarged portion 705 include the I/E phase signal 704, the airway pressure signal (Paw) 710, the slope of the net flow signal (slopeQnet1) 726, a second slope of the net flow signal (2ndSlopeQnet1) 728, and a delayed-cycling signal 716.

The second slope of the net flow signal (2ndSlopeQnet1) 728 represents a change in the slope of the net flow signal (slopeQnet1). The second slope of the net flow signal (2ndSlopeQnet1) 728 may be considered to be similar to the acceleration of the net flow signal (Qnet1). The second slope of the net flow signal (2ndSlopeQnet1) 728 may be determined or calculated by subtracting a current slopeQnet1 value from a prior slopeQnet1 value. For example, the 2ndSlopeQnet1 signal 728 may be calculated using the following Equation 5:

2ndSlopeQnet1=slopeQnet1(k)−slopeQnet1(k−μ) (Eqn. 5)

In Equation 5, k represents a current measurement cycle or control cycle and μ is a constant that represents a number of prior measurement cycles. In some examples, the constant μ may be the same as the constant α. In other examples, the constant μ may be less than the constant α. For instance, the constant μ may be between 1-15, and the resultant time duration may be between 10 ms and 80 ms.

In the plots of FIGS. 7A-7B, an occurrence of a delayed-cycling asynchrony event is highlighted by the circle 718. Delayed-cycling occurs when the exhalation valve opens too late causing mechanical inspiration to extend into the neural expiration. This may unnecessarily increase the delivered tidal volume and shorten the available expiratory time to promote dynamic hyperinflation. In a delayed-cycling event where a mechanical inspiration is longer than the patient's neural inspiration, the patient's expiratory effort pushes the flow out of the lungs, which results in increasing end-inspiratory pressure and descending 2^{nd} slope of Qnet1 (shown at Point “c” in the enlarged portion 705) until the neural inhalation ends.

In some examples, the following criteria may be used to determine whether a delayed-cycling asynchrony event has occurred. Specifically, if the following three criteria are met, a delayed-triggering asynchrony event is declared and the delayed-cycling signal 716 goes to high/true:

Example Delayed-Cycling Asynchrony Detection Criteria:
(1) breath phase is inspiration phase;
(2) inspiration time has passed duration threshold (γ); and
(3) the value of the value of 2ndSlopeQnet1 goes below second slope threshold (w).

The duration threshold (φ) in Criterion 2 is time threshold. In some examples, the duration threshold (φ) may be between 50 ms and 500 ms, such as 100 ms, 150 ms, 200 ms, 250 ms, etc. The second slope threshold (ψ) may be between −1.0 and −6.0 Liter/min³.

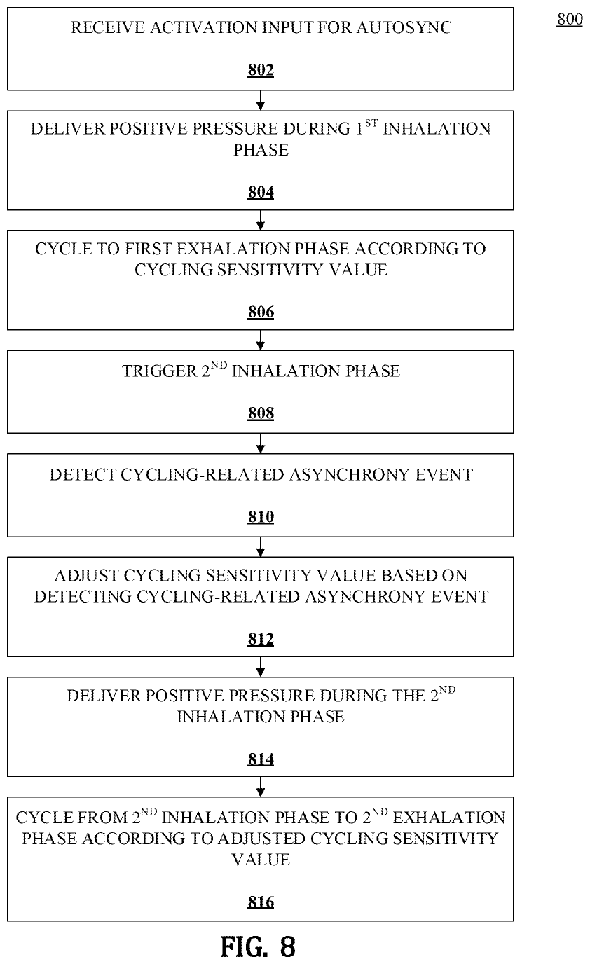

FIG. 8 depicts an example method 800 for automatic synchronization in medical ventilation. The operations of method 800 may be performed by a ventilator and/or a component thereof. At operation 802, a user input is received to activate an automated synchronization mode. The input may be received via a touch screen of the ventilator or via some other input mechanism of the ventilator. In some examples, this step may be omitted and the ventilator, or a particular mode of the ventilator, may automatically utilize automated synchronization operations.

At operation 804, positive pressure is delivered during a first inhalation phase. The positive pressure may be delivered according to a ventilation type or ventilation strategy set on the ventilator, which may be selected by the user. At operation 806, the ventilator cycles to a first exhalation phase at the end of the first inhalation phase. The cycling to the first exhalation phase is performed according to a cycling sensitivity of the ventilator. The cycling sensitivity may be represented or controlled by a cycling sensitivity value or setting, as discussed above. At the beginning of ventilation, the cycling sensitivity value may be a default value used by the ventilator. In other examples, the cycling sensitivity value may be an initial value that is set based on user input. At operation 808, a second inhalation phase is triggered at the end of the first exhalation phase.

At operation 810, a cycling-related asynchrony event is detected. The cycling-related asynchrony event may be detected during at least one of the first exhalation phase or the second inhalation phase. For example, different types of cycling-related asynchrony events may be detected in different phases, as discussed above. The detection of the cycling-related asynchrony event may be based on the types of factors and criteria discussed above. For instance, the cycling-related asynchrony may be detected based on measured flow signals, a Psync signal, or other types of signals, as discussed above. As an example, the cycling-related asynchrony event may be detected based on a slope of a net flow crossing zero during the first exhalation phase. Alternatively or additionally, the cycling-related asynchrony event may be detected based on a slope of a net flow crossing zero during the first exhalation phase. Further, the cycling-related asynchrony event may be detected based on a slope of a net flow exceeding a slope threshold during the first exhalation phase. While the term “exceeding” is used herein, such a term also applies to a negative crossing of a negative threshold (e.g., a signal moving further negative passing a negative threshold). As another option, the cycling-related asynchrony event may be detected based on a second slope of a net flow exceeding a slope threshold during the first exhalation phase. Further, the cycling-related asynchrony event may be detected based on a Psync signal being negative during an exhalation phase. Other factors and criteria discussed above, along with the combinations thereof, may also be used in detecting the cycling-related asynchrony events.

Based on detecting the cycling-related asynchrony event in operation 810, the cycling sensitivity of the ventilator is adjusted in operation 812. Adjusting the cycling sensitivity of the ventilator may include adjusting a cycling sensitivity value or setting. Adjusting the cycling sensitivity value may be based on the type of the asynchrony event that is detected. For example, for some cycling-related asynchrony events (e.g., a delayed-cycling event), the cycling sensitivity may be increased. For other cycling-related asynchrony events (e.g., a premature-cycling event or a double-triggering event), the cycling sensitivity may be decreased. The amount of the adjustment to the cycling sensitivity may also be based on the type of cycling-related asynchrony event that is detected. The adjustment to the cycling sensitivity may also be performed automatically by the ventilator without any additional user interaction or input.

At operation 814, positive pressure is delivered by the ventilator during the second inhalation phase. At operation 816, the ventilator cycles from the second inhalation phase to the second exhalation phase according to the adjusted cycling sensitivity that resulted from the adjustment made in operation 812. Method 800 may continue to monitor for additional or subsequent asynchrony events and continue to adjust the cycling sensitivity while the patient is being ventilated. Accordingly, the ventilator is able to automatically adjust as the patient's condition changes.

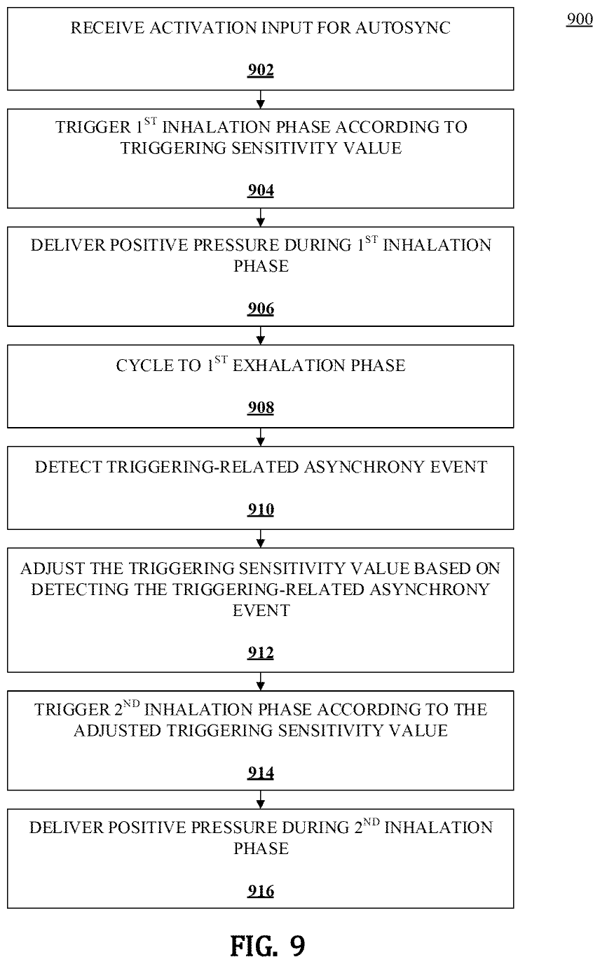

FIG. 9 depicts another example method 900 for automatic synchronization in medical ventilation. Method 900 is similar to method 800 with the primary exception that method 900 is related to triggering-related asynchronies rather than cycling-related asynchronies. Similar to method 800, at operation 902 method 900 receives a user input for activating automated synchronization.

At operation 904, a first inhalation phase is triggered according to a triggering sensitivity of the ventilator. The triggering sensitivity may be represented or controlled by a triggering sensitivity value or setting, as discussed above. At the beginning of ventilation, the triggering sensitivity value may be a default value and/or a user-set value when initializing the automated synchronization mode. At operation 906, positive pressure is delivered during the first inhalation phase. At operation 908, the ventilator cycles to a first exhalation phase at the end of the first inhalation phase.

At operation 910, during the first exhalation phase, a triggering-related asynchrony event is detected. Detecting the triggering-related asynchrony event may be based on the factors and/or criteria discussed above. For example, the triggering-related asynchrony event may be detected based on a local minimum and a local maximum of a slope of net flow signal. As another example, the first triggering-related asynchrony event is detected based on a local minimum and a local maximum of a slope of a derivative of an intrapleural pressure signal (e.g., slopePsync). Other criteria discussed above may also be used detecting a triggering-related asynchrony event.

At operation 912, in response to detecting the triggering related asynchrony event in operation 810, the triggering sensitivity is adjusted. The ventilator may automatically adjust the triggering sensitivity without any additional user input or interaction. Adjusting the triggering sensitivity of the ventilator may be accomplished by adjusting a triggering sensitivity value or setting of the ventilator. Adjusting the triggering sensitivity may also be based on the type of the asynchrony event that is detected. For example, for some triggering-related asynchrony events (e.g., an auto-triggering event), the cycling sensitivity is decreased, which causes the ventilator to be less sensitive to triggering (e.g., a greater patient effort is required to trigger a breath). In some examples, decreasing the triggering sensitivity may include increasing the triggering sensitivity value or threshold. For other triggering-related asynchrony events (e.g., a missed-trigger event), the cycling sensitivity is increased, which causes the ventilator to more sensitive to triggering (e.g., a weaker patient effort will trigger a breath). In some examples, increasing the triggering sensitivity may include decreasing the triggering sensitivity value or threshold. The amount of the adjustment to the cycling sensitivity may also be based on the type of triggering-related asynchrony event that is detected.

At operation 914, at the end of the first exhalation phase, a second inhalation phase is triggered according to the adjusted triggering sensitivity that resulted from the adjustment made in operation 912. At operation 916, positive pressure is delivered during the second inhalation phase.

Method 900 may continue to monitor for additional or subsequent asynchrony events and continue to adjust the triggering sensitivity while the patient is being ventilated. For example, method 900 may further include cycling from the second inhalation phase to a second exhalation phase, and then, during the second exhalation phase, detecting a second triggering-related asynchrony event. Based on detecting the second triggering-related asynchrony event, the ventilator may then re-adjust (e.g., a second adjustment) the adjusted triggering sensitivity. The amount of the first adjustment may differ from the amount of the second adjustment. For example, the first triggering-related asynchrony event may be an auto-triggering event, and the second triggering-related asynchrony event is a missed-triggering event. In such an example, the amount of the first adjustment may be less than the amount of the second adjustment. In addition, the method 900 may be combined with (or executed concurrently with) method 800 to allow for changes to both the triggering sensitivity values and the cycling sensitivity values. Accordingly, the ventilator is able to automatically adjust as the patient's condition changes.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method, performed by a ventilator, for automatic synchrony adjustment in medical ventilation, the method comprising:
delivering positive pressure during a first inhalation phase;
cycling to a first exhalation phase at an end of the first inhalation phase according to a cycling sensitivity of the ventilator;
at an end of the first exhalation phase, triggering a second inhalation phase;
during at least one of the first exhalation phase or the second inhalation phase, detecting a cycling-related asynchrony event;
in response to the detecting, automatically adjusting the cycling sensitivity without additional user input;
delivering positive pressure during the second inhalation phase; and
cycling from the second inhalation phase to a second exhalation phase according to the adjusted cycling sensitivity.

2. The method of claim 1, wherein the cycling-related asynchrony event is detected during the first exhalation phase.

3. The method of claim 1, wherein the cycling-related asynchrony event is detected during the second inhalation phase.

4. The method of claim 1, wherein the cycling-related asynchrony event is one of a premature-cycling event or a double-triggering event, and adjusting the cycling sensitivity decreases the cycling sensitivity.

5. The method of claim 1, wherein the cycling-related asynchrony event is a delayed-cycling event, and adjusting the cycling sensitivity increases the cycling sensitivity.

6. The method of claim 1, wherein detecting the cycling-related asynchrony event comprises a slope of a net flow crossing zero during the first exhalation phase.

7. The method of claim 1, wherein detecting the cycling-related asynchrony event comprises at least one of a slope of a net flow exceeding a slope threshold during the first exhalation phase, or a second slope of a net flow exceeding a slope threshold during the first exhalation phase.

8. The method of claim 1, further comprising, prior to detecting a cycling-related asynchrony event, receiving a user input to activate an automated synchronization mode.

9. A method, performed by a ventilator, for automatic synchrony adjustment in medical ventilation, the method comprising:
triggering a first inhalation phase according to a triggering sensitivity of the ventilator;
delivering positive pressure during the first inhalation phase;
cycling to a first exhalation phase at an end of the first inhalation phase;
during the first exhalation phase, detecting a missed-triggering event associated with the first inhalation phase;
in response to detecting the missed-triggering event, automatically increasing the triggering sensitivity without additional user input;
at an end of the first exhalation phase, triggering a second inhalation phase according to the increased triggering sensitivity; and
delivering positive pressure during the second inhalation phase.

10. The method of claim 9, further comprising, subsequent to the first exhalation phase, detecting an auto-triggering event, and in response automatically decreasing the triggering sensitivity without additional user input.

11. The method of claim 10, wherein increasing the triggering sensitivity comprises increasing by a first amount, and decreasing the triggering sensitivity comprises decreasing by a second amount, and wherein the second amount is different than the first amount.

12. The method of claim 11, wherein the second amount is less than the first amount.

13. The method of claim 9, wherein the missed-triggering event is detected based on a local minimum and a local maximum of a slope of a net flow signal.

14. The method of claim 9, wherein the missed-triggering event is detected based on a local minimum and a local maximum of a slope of a derivative of an intrapleural pressure signal.

15. The method of claim 9, wherein the missed-triggering event is detected based on:
a local minimum and a local maximum of a slope of a derivative of an intrapleural pressure signal; and
a local minimum and a local maximum of a slope of a net flow signal.
